(19) United States
(12) Patent Application Publication
(10) Pub. No.: US 2025/0177057 A1
(43) Pub. Date: Jun. 5, 2025

(54) SURGICAL NAVIGATION SYSTEM AND METHOD THEREOF

(71) Applicants: YI-ZENG HSIEH, Taipei (TW); MIN-CHUN HU, Hsinchu (TW); FU JEN CATHOLIC UNIVERSITY, New Taipei (TW)

(72) Inventors: SHU-HAO CHANG, New Taipei (TW); YI-ZENG HSIEH, Taipei (TW); CHENG-HOU CHOU, Taipei (TW); MIN-CHUN HU, Hsinchu (TW); HUNG-KUO CHU, Hsinchu (TW); PIN-XUAN LIU, Hsinchu (TW); CHUNG-LIANG HUANG, Hsinchu (TW)

(21) Appl. No.: 18/842,794

(22) PCT Filed: Mar. 2, 2023
(86) PCT No.: PCT/CN2023/079326

§ 371 Date: Aug. 29, 2024

Related U.S. Application Data
(60) Provisional application No. 63/315,546, filed on Mar. 2, 2022.

(51) Int. Cl. A61B 34/20; A61B 90/00; A61B 90/50; A61F 2/46

(52) U.S. Cl. CPC A61B 34/20; A61B 90/39; A61F 2/4609; A61B 2034/2057; A61B 2034/2065; A61B 2034/207; A61B 2090/373; A61B 2090/3916; A61B 2090/3937; A61B 2090/502

(57) ABSTRACT

A surgical navigation system (100) is provided. The surgical navigation system (100) includes: a head-mounted device (110), which includes a sensor module (111), a processing module (112), and a display module (113); and a plurality of visual markers (120), wherein three-dimensional position and orientation of each visual marker (120) is recognized and tracked by the sensor module (111), and then the processing module (112) calculates spatial conversion relationship between each visual marker (120) to create a local coordinate system, and then the display module (113) generates a virtual image. A method of using the surgical navigation system (100) to assist medical procedure is also provided.

### CROSS-REFERENCE TO RELATED APPLICATIONS

This application claims the benefit of and priority to U.S. Provisional Patent Application Ser. No. 63/315,546, entitled “SYSTEMS AND METHODS FOR SURGICAL NAVIGATION BASED ON MIXED REALITY”, filed on Mar. 2, 2022. The contents of the above-mentioned application are hereby incorporated by reference herein for all purposes.

### FIELD

The present disclosure generally relates to a surgical navigation system and method thereof, more particularly, to a surgical navigation system and method for assisting medical procedures.

### BACKGROUND

Lewinnek et al. defined the “Lewinnek Safe Zone” for an acetabular component to avoid risks of joint dislocation during a hip joint replacement surgery. If the acetabular component is not placed within the safety zone, the risks of dislocation, pinching, limited mobility, early loosening, and wear of a polyethylene component of the artificial hip joint is greatly increased.

However, to know the suitable placement angles of the acetabular component, most post-operative X-rays are required to measure whether the acetabular component is within the safety zone. If a navigation system is not used, only traditional instruments could be used to guide, and there are still many human errors due to intraoperative displacement of a pelvis or differences in a interpretation of instruments.

Therefore, there is an urgent need to develop a method and system that could instantly calculate and visually display the safety zone of the acetabular component placement without additional handheld analysis software or hardware and that is applicable to all brand-name machines and instruments to improve the efficiency of the artificial hip joint replacement surgery.

### SUMMARY

In view of the shortcomings in the art, some embodiments of the present disclosure may provide a surgical navigation system with a calibration procedure mode to make the surgical navigation system be well applied on any tools or instruments with different dimension.

Also, some embodiments of the present disclosure may provide a surgical navigation system with a hip replacement procedure mode to assist accurately inserting an acetabular component in a safe zone, or other medical procedure modes to assist the medical procedures.

In one aspect of the present disclosure, a surgical navigation system, comprising: a head-mounted device, comprising: a sensor module, comprising at least one tracking camera; a processing module, connected to the sensor module; and a display module, connected to the processing module, and comprising a display generator; and a plurality of visual markers, recognized and tracked individually by the tracking camera; wherein, three-dimensional position and orientation of each of the plurality of visual markers is recognized and tracked by the tracking camera, and then the processing module calculates spatial conversion relationship between each of the plurality of visual markers based on the three-dimensional position and orientation to create a local coordinate system, and then the display module generates a virtual image based on the local coordinate system through the display generator.

According to an implementation of the first aspect, one of the visual markers is used as a positioning reference that includes a calibration part, and the positioning reference is fixed on body of a patient or stays still when the surgical navigation system is used for a medical procedure.

According to another implementation of the first aspect, the surgical navigation system further comprises a registration pointer that is attached with at least one visual marker and includes a registering part.

According to another implementation of the first aspect, when the registering part of the registration pointer points the calibration part of the positioning reference, the processing module calculates spatial conversion relationship between the registration pointer and the positioning reference based on the three-dimensional position and orientation of the visual markers to calibrate a distance between the registering part and the visual marker on the registration pointer.

According to another implementation of the first aspect, the registration pointer is used to register three-dimensional position and orientation of one or more landmarks on the patient.

According to another implementation of the first aspect, the registration pointer is used to calibrate a distance between a surgical instrument and a visual marker thereon through pointing boundary of the surgical instrument with the registering part.

According to another implementation of the first aspect, the surgical instrument is an impactor with an acetabular component for hip replacement surgery, and the registration pointer is used to calibrate a distance between the acetabular component of the impactor and the visual marker thereon through pointing two ends of the impactor with an acetabular component with the registering part.

According to another implementation of the first aspect, the medical procedure is selected from the group consisting of hip replacement surgery, knee replacement surgery, corrective osteotomy for malunion of an arm bone, distal femoral and proximal tibial osteotomy, peri-acetabular osteotomy, elbow ligament reconstruction, knee ligament reconstruction, ankle ligament reconstruction, shoulder acromioclavicular joint reconstruction, total shoulder replacement, reverse shoulder replacement, total ankle arthroplasty.

In another aspect of the present disclosure, a method of using the surgical navigation system to assist a hip replacement surgery, comprising: fixing a first visual marker as the positioning reference on the patient; attaching a second visual marker on the registration pointer; recognizing the first and the second visual markers individually and pointing the calibration part of the positioning reference by the registering part of the registration pointer points to calibrate a distance between the registering part and the visual marker on the registration pointer; pointing one or more landmarks of pelvis of the patient by the registering part of the registration pointer to register three-dimensional position and orientation of the one or more landmarks; defining a local coordinate system based on the three-dimensional position and orientation of the one or more landmarks; attaching a third visual marker on femur of the patient; recognizing the third visual markers and move the femur of the patient horizontally and vertically to determine a center of a hip joint; and defining a safe zone for being inserted with an acetabular component based on the local coordinate system and the center of the hip joint.

According to an implementation of the second aspect, the one or more landmarks comprises a left anterior superior iliac spine (ASIS), a right ASIS, and a pubic symphysis, and the local coordinate system is related to a pelvis size or position and orientation of an anterior pelvic plane.

According to another implementation of the second aspect, the method further comprises: attaching a fourth visual marker on an impactor with the acetabular component; and recognizing the second and the fourth second visual markers individually and point two ends of the impactor with an acetabular component by the registering part of the registration pointer points to calibrate the distance between the acetabular component of the impactor and the fourth visual marker.

According to another implementation of the second aspect, the method further comprises: tracking and guiding the impactor with the acetabular component to align the safe zone.

The surgical navigation system of the present disclosure could visually display the safe zone of the acetabular component placement instantly and accurately, and could be applicable to all brand-name machines and instruments to improve the efficiency of medical procedures by using the surgical navigation system of the present disclosure.

### BRIEF DESCRIPTION OF THE DRAWINGS

The present description will be better understood from the following detailed description when read in light of the accompanying drawings, where:

FIG. 1 is a schematic view of the surgical navigation system including a head-mounted device and a visual marker, in accordance with an embodiment of the present disclosure.

FIG. 2 is a schematic view of visual markers of the surgical navigation system, in accordance with an embodiment of the present disclosure.

FIG. 3 is a schematic view of the electrical hardware configuration of the surgical navigation system of FIG. 1, in accordance with the embodiment of the present disclosure.

FIG. 4 is a schematic view of using the surgical navigation system for hip replacement surgery, in accordance with an embodiment of the present disclosure.

FIG. 5 is a flowchart showing a method of using the surgical navigation system to perform a calibration procedure, in accordance with an embodiment of the present disclosure.

FIG. 6 is a schematic view of a registration pointer of the surgical navigation system, in accordance with an embodiment of the present disclosure.

FIG. 7A and FIG. 7B are a flowchart showing a method of using the surgical navigation system to perform a hip replacement procedure, in accordance with an embodiment of the present disclosure.

FIG. 8 is a schematic view of an impactor of the surgical navigation system, in accordance with an embodiment of the present disclosure.

FIG. 9 is a schematic view of displayed images of the surgical navigation system, in accordance with an embodiment of the present disclosure.

### DETAILED DESCRIPTION

The following disclosure contains specific information pertaining to exemplary embodiments in the present disclosure. The drawings in the present disclosure and their accompanying detailed disclosure are directed to merely exemplary embodiments. However, the present disclosure is not limited to merely these exemplary embodiments. Other variations and embodiments of the present disclosure will occur to those skilled in the art. Unless noted otherwise, like or corresponding elements among the figures may be indicated by like or corresponding reference numerals. Moreover, the drawings and illustrations in the present disclosure are generally not to scale and are not intended to correspond to actual relative dimensions.

For the purposes of consistency and ease of understanding, like features are identified (although, in some examples, not shown) by numerals in the exemplary figures. However, the features in different embodiments may be different in other respects, and thus shall not be narrowly confined to what is shown in the figures.

Terms such as “at least one embodiment”, “one embodiment”, “multiple embodiments”, “different embodiments”, “some embodiments,” “present embodiment”, and the like may indicate that an embodiment of the present invention so described may include a particular feature, structure, or characteristic, but not every possible embodiment of the present invention must include a particular feature, structure, or characteristic. Furthermore, repeated use of the phrases “in one embodiment”, “in this embodiment”, and so on does not necessarily refer to the same embodiment, although they may be identical. Furthermore, the use of phrases such as “embodiments” in connection with “the present invention” does not imply that all embodiments of the present invention necessarily include a particular feature, structure, or characteristic, and should be understood as “at least some embodiments of the present invention” include the particular feature, structure, or characteristic described. The term “coupled” is defined as connected, directly or indirectly through intervening components, and is not necessarily limited to physical connections. The term “comprising” refers to “including but not necessarily limited to”, which specifically indicates open-ended inclusion or membership in the so-described combination, group, series, and the equivalent.

Additionally, for the purposes of explanation and non-limitation, specific details such as functional entities, techniques, protocols, standards, and the like are set forth for providing an understanding of the described technology. In other examples, detailed disclosure of well-known methods, technologies, systems, architectures, and the like are omitted so as not to obscure the disclosure with unnecessary details.

The terms “first”, “second”, and “third” in the description of the present invention and the above-mentioned drawings are used to distinguish different objects, rather than to describe a specific order. Furthermore, the term “comprising” and any variations thereof are intended to cover non-exclusive inclusions. For example, a process, method, system, product, or device that includes a series of steps or modules is not limited to the listed steps or modules, but optionally also includes steps or modules that are not listed, or optionally also includes other steps or modules that are inherent to those processes, methods, products, or devices.

The present invention will be described in further detail below in conjunction with the accompanying drawings and embodiments.

The present disclosure is generally related to a surgical navigation system and method thereof for providing intuitive real time guide images to assist medical procedures.

The surgical navigation system and method thereof may be used by a user to assist a medical procedure, including surgical operations, on a human or mammal (e.g., another patient). In some embodiments, the user may be an individual using the surgical navigation system and method thereof of the present disclosure. In some embodiments, the patient may be another individual who may be a subject of a medical procedure performed by the surgical navigation system and method thereof of the present disclosure.

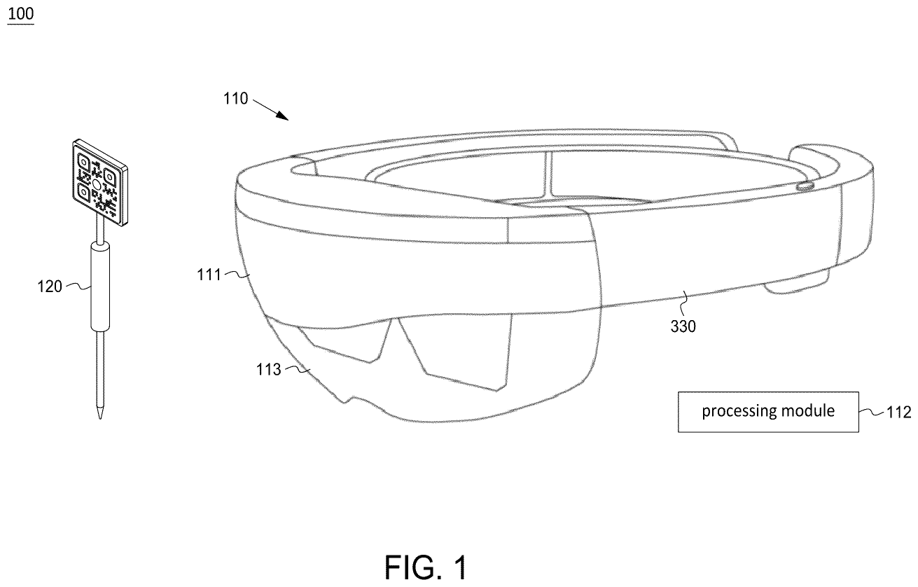

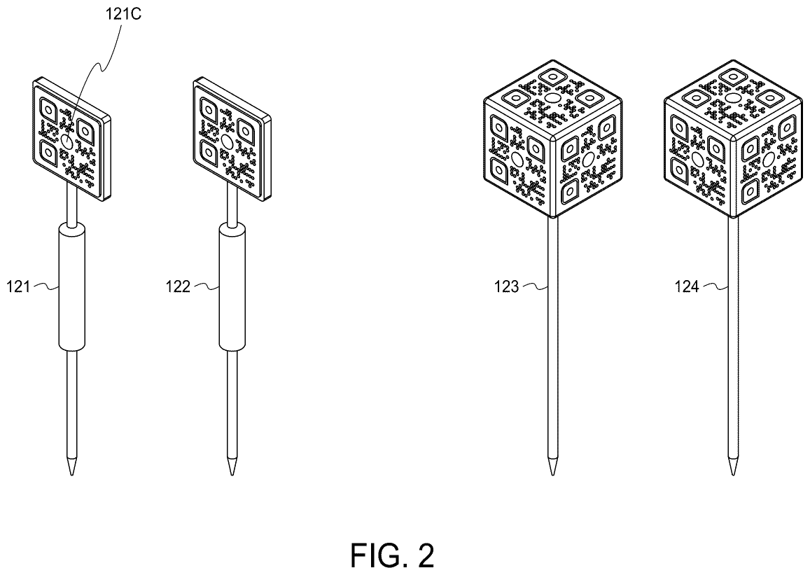

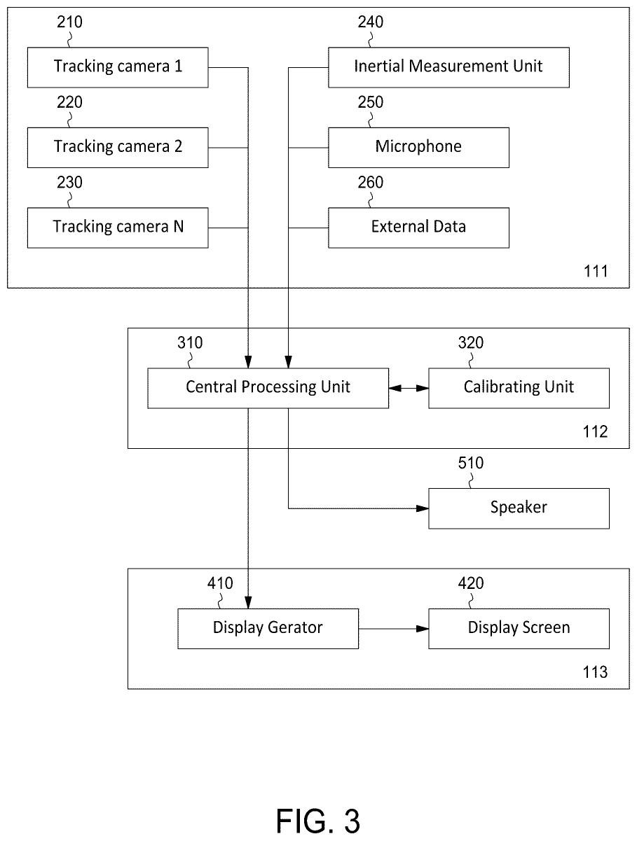

Referring to FIG. 1, FIG. 2, and FIG. 3, a surgical navigation system 100 of the present disclosure is provided for assisting medical procedures; wherein, FIG. 1 is a schematic view of the surgical navigation system including a head-mounted device 110 and a visual marker 120, in accordance with an embodiment of the present disclosure; FIG. 2 is a schematic view of visual markers (121, 122, 123, 124) of the surgical navigation system, in accordance with an embodiment of the present disclosure; and FIG. 3 is the electronic hardware configuration of the surgical navigation system 100, in accordance with the embodiment of the present disclosure.

The surgical navigation system 100 may include a head-mounted device 110, and one or more visual markers 120. In some embodiments, the head-mounted device 110 may include a sensor module 111 having one or more tracking cameras (210, 220, 230), a processing module 112, and a display module 113 having a display generator 410 that generates a visual display on a display screen 420 for viewing by a user. In one preferred embodiment, the display module 113 is attached to the user's head, more specifically, the display screen 420 is arranged in front of the user's eyes. In some embodiments, the processing module 112 may further include a calibrating unit 320.

In some embodiments, the sensor module 111, the processing module 112, and the display module 113 may be incorporated together in the head-mounted device 110. In some embodiments, the processing module 112 may be remotely connected to the sensor module 111 and the display module 113 that are both deposited in the head-mounted device 110. The processing module 112 may be realized by a central processing unit (CPU) 310 or may be implemented by other programmable general-purpose or special-purpose microprocessor, digital signal processor (DSP), programmable controller, application specific integrated circuits (ASIC), programmable logic device (PLD), or other the like or any combinations thereof.

In some embodiments, the display screen 420 may include a clear face shield that allows a projection from the display generator 410 onto the clear face shield that overlays data and imagery within the visual path of the user's eyes. In some embodiments, the sensor module 111 may be attached or made part of the display module 113. In some embodiments, the display generator 410 may be in electronic communication with the display screen 420. In some embodiments, the display generator 410 may be incorporated together with the display screen 420 in the display module 113. In some embodiments, the display module 113, especially the display screen 420, may further include an attachment mechanism 330 that allows attachment to the user's head or face such that the alignment of the display module 113 to the user's visual path is consistent and repeatable.

Referring to FIG. 3, in some embodiments, the sensor module 111 may not only include one or more tracking cameras (210, 221, 230), but also optionally include an inertial measurement unit (IMU) 240, a microphone 250 for voice activation of different display modes, including but not limited to removal of all displayed items for a clear field of view, and one or more speakers 510 for audible alerts and other purposes.

In some embodiments, the IMU 240 may be provided added orientation and localization data for an object that is not visually based. In some embodiments, the sensor module 111 may further include external data 260 as relayed by wire, radio or stored memory, and the external data 260 may optionally be in the forms of fluoroscopy imagery, computerized axial tomography (CAT or CT) scans, positron emission tomography (PET) scans, magnetic resonance imaging (MRI) data, or the like.

In some embodiments, during operation of the surgical navigation system 100, the display generator 410 and the processing module 112 are in electronic communication with the components described above for the sensor module 111. The processing module 112 may be a central processing unit (CPU) 310 that controls display management and algorithm prosecution. In some embodiments, the processing module 112 may be a combination of a CPU 310 that controls display management and algorithm prosecution and a calibrating unit 320 that controls calibration of orientation and localization data. In some embodiments, the processing module 112 may be a CPU 310 that has a calibrating sub-unit to controls calibration of orientation and localization data.

In some embodiments, the surgical navigation system 100 may use one or more sensor module 111 to create a cloud of three-dimensional point data representing objects in a workspace. This data may be used to create or map to modeled objects for follow-up, visualization, or playback at a later time. In some embodiments, the display module 113 may include, but not be limited to, holographic or pseudo holographic display projection into the field of regard for the user. Furthermore, the display module 113 may optionally provide disclosed means of eye tracking that allows determination of the optimal displayed imagery with respect to the user's visual field of view.

In some embodiments, the surgical navigation system 100 may optionally use algorithms to discriminate between items in the field of view to identify what constitutes objects of interest versus objects not important to the task at hand. This may include, but is not limited to, identifying bony landmarks on a hip acetabulum for use in comparison and merge with a pre-operative scan in spite of soft tissue and tools that are visible in the same field of regard.

In some embodiments, the display module 113 may be realized by an AR head-mounted device. The AR head-mounted device may be used in various sterile surgical procedures (e.g., spinal fusion, hip and knee arthroplasty, etc.). The AR head-mounted device may be clamped on the head of the user by adjusting a head strap by turning a thumb wheel. Furthermore, a transparent protective face shield may be optionally attached to the AR head-mounted device by attachment to Velcro strips. Alternatively, attachment may be via adhesive, magnetic, hooks, or other art-disclosed attachment means. In some embodiments, the AR head-mounted device may include a display section having a pair of display screen for visual augmentation and two tracking cameras for performing tracking and stereoscopic imaging functions including two-dimensional and three-dimensional digital zoom functions. Alternatively, the display module 113 may be realized by an MR head-mounted device.

In some embodiments, the one or more tracking cameras (210, 220, 230) of the sensor module 111 and the one or more visual markers (121, 122, 123, 124) are used to visually track a distinct object (e.g., a surgical tool, a desired location within an anatomical object, etc.) and determine attitude, position, and orientation relative to the user.

In some embodiments, the one or more visual markers (121, 122, 123, 124) may be recognized by the one or more tracking cameras (210, 220, 230) of the sensor module 111. In some embodiments, each of the one or more visual markers (121, 122, 123, 124) is distinct and different from each other visually, and thus the one or more visual markers (121, 122, 123, 124) may be individually tracked by the one or more tracking cameras (210, 220, 230). Standalone object recognition and machine vision technology may be used for marker recognition. In some embodiments, the one or more visual markers may be a 1D barcode or a 2D barcode, such as QR code, PDF 417, or the like.

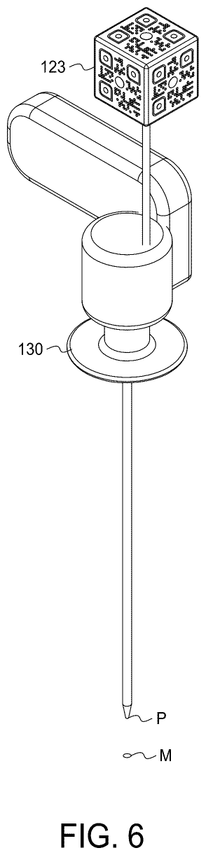

In some embodiments, at least one of the visual markers 121 may be attached on a locator as a positioning reference that contains a calibration point/part 121C to perform a calibration procedure. The locator may be a bone pin, clamp, or any tools that could be fixed on the body of the patient. In some embodiments, the calibration point 121C may be deposited on a center of the visual marker. In some embodiments, the one or more visual markers 123 may be attached on a registration pointer 130 (shown as FIG. 6) or any types of surgical instruments to track position and orientation of them. In some embodiments, the one or more visual markers 122 may be attached on body of the patient to track position and orientation of the patient. In some embodiments, the visual markers (121, 122, 123, 124) may be deposited on any means as long as the visual marker (121, 122, 123, 124) could be recognized and tracked by the tracking cameras (210, 220, 230); for example, the visual markers (121, 122, 123, 124) may be deposited on a cube, a cuboid, a trigonal trapezohedron. In one preferred embodiment, the visual markers (121, 122, 123, 124) may be deposited on five faces of a cube to increase possibilities of recognized and tracked by the tracking cameras (210, 220, 230).

The present disclosure may be used for surgical procedures. In some embodiments, a pre-operative planning may be performed (optionally using AR or MR for visualization and manipulation of models) using models to identify items including but not limited to: anatomic reference frames, targets for resection planes, volumes to be excised, planes and levels for resections, size and optimum positioning of implants to be used, path and trajectory for accessing the target tissue, trajectory and depth of guidewires, drills, pins, screws or instruments. In some embodiments, the models and pre-operative planning data may be uploaded into the memory of the display module 113 prior to or at time of surgery, wherein the uploading process may most conveniently be performed wirelessly via the radio.

Algorithms in the AR head-mounted device may be used to process the images from the one or more tracking cameras (210, 220, 230) to calculate the point of intersection of each fiduciary and thereby determine the six-degrees of freedom pose of the visual markers (121, 122, 123, 124). The “pose” herein refers to the combination of position and orientation of an object. In some embodiments, fiducials of the visual markers (121, 122, 123, 124) may be created by printing on self-adhesive sticker, by laser-etching the black regions onto the surface of white plastic material or alternative methods.

In some embodiments, the user may insert the one or more visual markers (121, 122, 123, 124) into a bone of the patient for precise tracking. In some embodiments, when the user using the surgical navigation system 100 with the display module 113 during surgery, the user may see the pre-operative planning information and may track surgical instruments and implants and provide intraoperative measurements of various sorts including but not limited to depth of drill or screw relative to anatomy, angle of an instrument, angle of a bone cut, etc.

In some embodiments, when the surgical navigation system 100 is used during a medical procedure, the processing module 112 may be booted, and the one or more tracking cameras (210, 220, 230) may be initialized. The positioning reference (e.g., the visual marker 121 attached on the locator) may be located and identified followed by the subsequent visual markers (121, 122, 123, 124) when they are in the field of view of the one or more tracking cameras (210, 220, 230). The track of these visual markers (121, 122, 123, 124) may provide position and orientation relative to each other. Alternate sensor data from the sensor module 111 such as IMU 240 may be optionally incorporated into the data collection. Further, external (assistance) data 260 about the patient, target, tools, instruments, or other portions of the environment may be optionally incorporated for use in the algorithms. The algorithms used in the present disclosure may be tailored for specific procedures and data collected. The algorithms may output the desired assistance data for use in the display module 113.

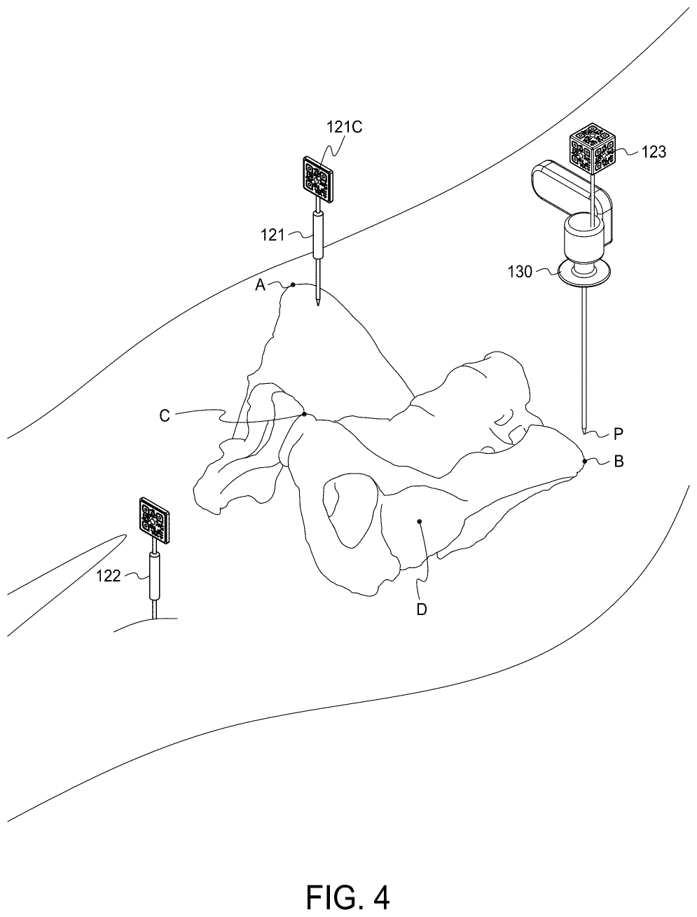

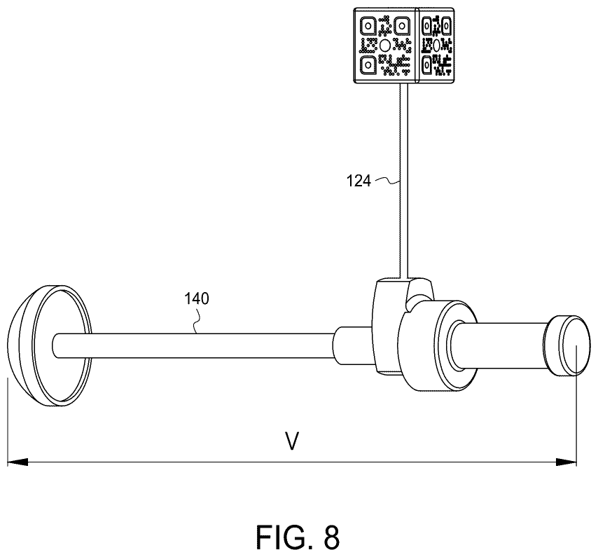

In one exemplary embodiment of the present disclosure, referring to FIG. 4, the surgical navigation system 100 may be used for hip replacement surgery. The visual marker 121 may be attached on the locator that is fixed on a pelvis of the patient as a positioning reference, another visual marker 123 may be attached on a registration pointer 130, other visual markers 124 may be attached on surgical instruments, such as an impactor 140 (shown as FIG. 8), and the other visual markers 122 may be attached on body of the patient, such as on skin or femur. In some embodiments, the dimensions of the registration pointer 130 and the impactor 140 may be known or unknown. In one preferred embodiment, the visual marker 122 may be attached on the femur of the patient, and the visual marker 124 may be attached on the impactor 140.

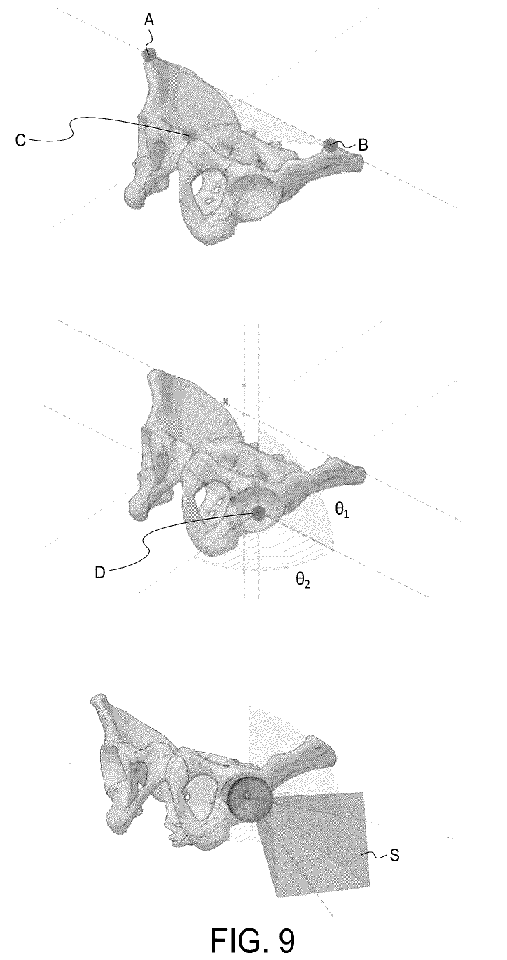

The user may see the mixed reality user interface image (MRUI) shown in FIG. 9 via the display module 113, which provides stereoscopic virtual images of a safe zone(S) for inserting a hip cup (i.e., an acetabular component) in the user's field of view during the hip replacement procedure. The safe zone herein refers to a safer range to insert the acetabular component, and the safe zone may cover a range of abduction (inclination) for 40±10 degree and anteversion for 15±10 degree from a hip center or from a center of acetabular component surface. Further, when the surgical navigation system 100 is used for hip replacement surgery, the cover range of abduction and anteversion from the hip center or the center of acetabular component surface may be adjusted based on situations of patients and the acetabular component.

The combination of the one or more visual markers (121, 122, 123, 124) on these physical objects, combined with the prior processing and specific algorithms allows calculation of measures of interest to the user, including real time anteversion and inclination angles of the impactor with respect to the pelvis for accurate placement of acetabular shell (same as the acetabular component). Further, measurements of physical parameters from pre-to post-operative states may be presented, including but not limited to change in overall leg length. Presentation of data may be in readable form or in the form of imagery including, but not limited, to 3D representations of tools or other guidance forms, or any combinations thereof.

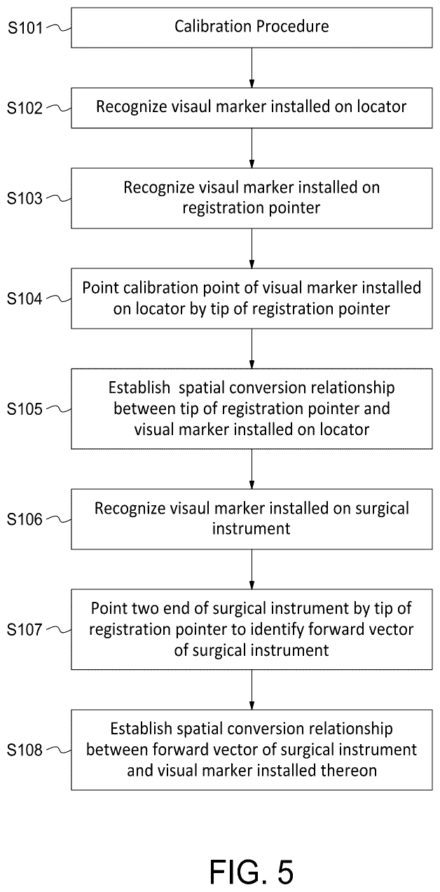

Referring to FIG. 4, FIG. 5, and FIG. 6, wherein FIG. 5 is a flowchart showing a method of using the surgical navigation system 100 to perform a calibration procedure, in accordance with an embodiment of the present disclosure, and FIG. 6 is a schematic view of a registration pointer of the surgical navigation system, in accordance with an embodiment of the present disclosure, respectively.

In some embodiments, the registration pointer 130 may form a roughly long rod and may be installed with one of the visual markers 123. In some embodiments, the registration pointer 130 may be used for the calibration procedure. In some embodiments, the registration pointer 130 may store one or more virtual pointing markers (M) that may be used to represent a position of interest to the user, including but not limit to bony landmarks. In some embodiments, the landmarks may be any points or combinations points on body of the patient that could be used to determine local coordinate system of a specific region interest to the user, such as pelvis of the patient. In some embodiments, the landmarks may be right anterior superior iliac spine (A), left anterior superior iliac spine (B), and pubic symphysis (C).

In some embodiments, before the surgical navigation system 100 of the present disclosure is used to assist the medical procedure during the hip replacement surgery, the registration pointer 130 and the surgical instruments may be calibrated by the calibration procedure. Therefore, the information of locations and orientations of the registration pointer 130 and each surgical instrument could be more accurate, and the registration pointer 130 and the surgical instruments with any dimensions would be applied in the surgical navigation system 100.

During the calibration procedure (S101), the visual marker 121 may be installed on the locator as the positioning reference that contains the calibration point 121C, and the locator may be fixed on a position related to regions interest to the user. In some embodiments, the locator may be fixed on the pelvis of the patient in the hip replacement surgery. The visual marker 121 installed on the locator may be recognized by the tracking cameras (210, 220, 230) of the sensor module 111 (S101), and the data related to three-dimensional position and orientation of the positioning reference may be transferred to and stored in the processing module 112, or the calibration unit 320. Another visual marker 123 may be installed on the registration pointer 130, which is used to be a main calibration tool during the calibration procedure. The visual marker 123 installed on the registration pointer 130 may be recognized by the tracking cameras (210, 220, 230) of the sensor module 111 (S102), and the data related to three-dimensional position and orientation of the registration pointer may be transferred to and stored in the processing module, or the calibration unit.

The calibration point 121C of the visual marker 121 installed on the locator may then be pointed by tip (P) of the registration pointer 130 (S103) when the visual markers (121, 123) are both recognized by the tracking cameras (210, 220, 230) to establish the spatial conversion relationship between the tip (P) of registration pointer 130 and visual marker 121 installed on locator by algorithm of the processing module 112, or the calibration unit 320 (S104). In some embodiments, the data related to the three-dimensional position and orientation of the registration pointer 130, especially the tip (P) thereof, may be transferred to and stored in the calibration unit 320 of the processing module 112 for being compared and calculated with the coordinate system of the positioning reference. More specifically, the calibration point 121C of the visual marker 121 is used to be an origin of coordinates, and when the tip (P) of the registration pointer 130 points the calibration point 121C of the visual marker 121, the three-dimensional position and orientation of the visual marker 123 related to the visual marker 121 may be recognized and tracked to calibrate the specific three-dimensional position and orientation between the tip (P) and the visual marker 123 of the registration pointer 130.

Other visual markers may be installed on surgical instruments, such as the impactor with the acetabular component. The visual markers installed on each surgical instrument may be recognized by the tracking cameras (210, 220, 230) of the sensor module 111 (S106), and the data related to three-dimensional position and orientation of the surgical instruments may be transferred to and store in the processing module 112. In one preferred embodiment, the visual marker 124 may be installed on the impactor 140 with the acetabular component (shown as FIG. 8). Two ends of each surgical instrument may then be pointed by the tip (P) of the registration pointer 130 when the visual markers (123, 124) are both recognized by the tracking cameras (210, 220, 230) to identify forward vector of each surgical instrument by algorithm of the processing module 112, or the calibration unit 320 (S107). In one preferred embodiment, the tip (P) of the registration pointer 130 points two ends or two positions along with an axis of the surgical instruments, and thus to identify forward vector of the surgical instrument more accurately. In another preferred embodiment, the tip (P) of the registration pointer 130 points two positions along with an axis of the impactor 140 with the acetabular component, and more specifically, the two positions along with the axis from the center the acetabular component to the impactor 140 to identify forward vector of the impactor 140 with the acetabular component more accurately. Based on the vector of each surgical instrument, the spatial conversion relationship between the forward vector of the surgical instrument and the visual markers 124 installed thereon may be established by algorithm of the processing module 112, or the calibration unit 320 (S108). In some embodiments, the data related to the three-dimensional position and orientation of each surgical instrument, especially the vector thereof, may be transferred to and stored in the calibration unit 320 of the processing module 112 for being compared and calculated with the coordinate system. More specifically, the three-dimensional position and orientation of each surgical instrument, especially the vector thereof, may be correlated with the visual marker 124 thereon. That is, the visual marker 124 may be used to be a parent element for storing the three-dimensional position and orientation of the surgical instrument.

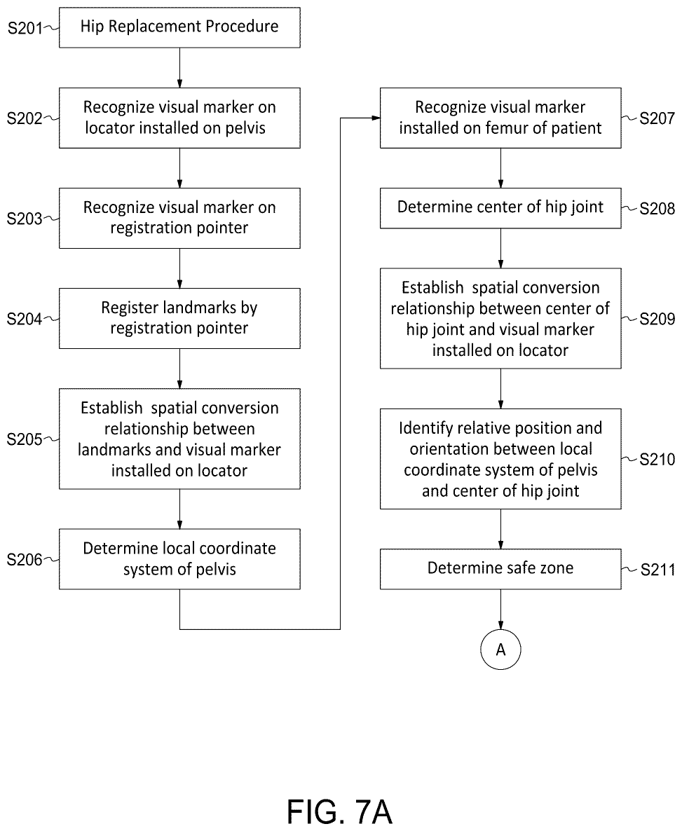

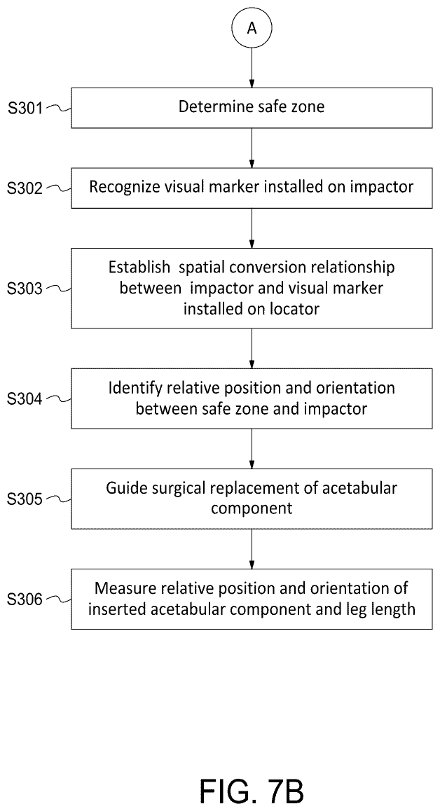

Referring to FIG. 7A and FIG. 7B, which are a flowchart showing a method of using the surgical navigation system 100 to perform a hip replacement procedure, in accordance with an embodiment of the present disclosure.

In some embodiments, the present disclosure may further provide a method of using the surgical navigation system 100 to perform a hip replacement procedure in which a hip bone has the socket reamed out and a replacement cup (e.g., acetabular component) is inserted for use with a patient's leg.

The visual marker 121 may be attached on the locator as a positioning reference, and the locator may be installed on pelvis of the patient. In some embodiments, the locator may be bone pins installed on pelvis of the patient. In some embodiments, the locator may be a clamp installed on pelvis of the patient. In some embodiments, the visual marker 121 may be attached on the locator by a clamp, Velcro, taps, and the like. In some embodiments, the visual marker 121 may be directly installed on body of the patient by a clamp, taps, or other art-disclosed attachment means. In some embodiments, the locator may be installed on any body parts of the patient as long as the visual marker 121 could be recognized by the one or more tracking cameras.

Another visual marker 123 may be attached on the registration pointer 130. In some embodiments, the visual marker 123 may be attached on the registration pointer 130 by a clamp, Velcro, taps, and the like. In some embodiments, the dimensions of the registration pointer 130 and a position or orientation of the visual marker 123 thereon may be unknown and need to be calibrated with the calibration procedure as described in FIG. 5. The registration pointer 130 may store the one or more virtual pointing markers (M) that may be used to register the position and orientation of landmarks interest to the user. In some embodiments, the landmarks may be any bony landmarks or other anatomic landmarks interest to the user. In some embodiments, the landmarks may be any points or combinations of points on body of the patient that could be used to determine a local coordinate system of pelvis. In some embodiments, the landmarks may be left anterior superior iliac spine (ASIS), right ASIS, and pubic symphysis, and thus each of them may be a virtual pointing marker of the registration pointer individually.

Other visual markers may be attached on femur of the patient. In some embodiments, the visual marker 122 may be attached on surface of the thigh (e.g., the skin of thigh). In some embodiments, the visual marker 122 may be attached on surface of the thigh by ac lamp, Velcro, taps (such as Ioban), or other art-disclosed attachment means.

When the surgical navigation system 100 of the present disclosure is used to assist hip replacement surgery with the hip replacement procedure (S201), the one or more visual markers (121, 122, 123) may be attached or installed as described above, and the registration pointer 130 may be calibrated with the calibration procedure. The visual marker 121 on the locater installed on pelvis of the patient may be recognized by the one or more tracking cameras (S202). The visual marker 123 on registration pointer 130 may also be recognized by the one or more tracking cameras (S203).

The position and orientation of the landmarks relative to the hip fixture may be registered by the tip (P) of the registration pointer 130 (S204) which may be viewed by the user on the display module. The position and orientation difference between the landmarks and the visual marker 121 installed on the locator may be calculated by the processing module 112 to establish spatial conversion relationship between the landmarks and the visual marker 12 installed on the locator (S205). Based on the established spatial conversion relationship, the local coordinate system of pelvis may be determined (S206), and real time guide markers for the local coordinate system of pelvis may be viewed on the display module 113. More specifically, the visual marker 121 installed on the locator may be used as a parent element of the landmarks, and the local coordinate system of pelvis would constantly follow the visual marker 121 installed on the locator when the patient moves. The preferent landmarks may include left, right ASIS, and pubic symphysis, and the local coordinate system of pelvis may include size of the pelvis size and the position and orientation of anterior pelvic plane.

The visual marker 122 on femur of the patient may also be recognized by the one or more tracking cameras (S207). The user may move the femur of the patient horizontally and vertically to determine center of the hip joint by art-disclosed means, such as least square sphere fit (S208). The position and orientation difference between the center of the hip joint and the visual marker 121 installed on the locator may be calculated by the processing module 112 to establish spatial conversion relationship between the center of the hip joint and the visual marker 121 installed on the locator (S209). More specifically, the visual marker 121 installed on the locator may be used as a parent element of the center of the hip joint, and the local coordinate system of the center of the hip joint would constantly follow the visual marker 121 installed on the locator when the patient moves.

Based on the spatial conversion relationship of the landmarks and the center of hip joint compared with the visual marker 121 installed on the locator, the relative position and orientation between local coordinate system of the pelvis and the center of the hip joint of the patient may be identified (S210). The safe zone may then be determined by 40±10 degree of abduction (inclination) angle and 15±10 degree of anteversion angle from the center of the hip joint (S211). The range(S) of the safe zone may be real-time displayed on the display module 113 as shown in FIG. 9. In some embodiment, the abduction (inclination) angle and anteversion angle may be adjusted according to different patients to display patient-specific safe zone on the display module. In some embodiments, the safe zone may then be determined by 40±10 degree of abduction (inclination) angle and 15±10 degree of anteversion angle from the center of the hip joint plus a thickness of the acetabular component.

Referring to FIG. 8, which is a schematic view of impactor 140 of the surgical navigation system 100, in accordance with an embodiment of the present disclosure. The surgical navigation system 100 may optionally include a hip impactor for use in hip arthroplasty procedures of the hip replacement surgery. The impactor 140 may include an acetabular shell (i.e., acetabular component) that may be inserted into the hip joint. In some embodiments, the user may directly control the impactor 140 to insert the acetabular component into the hip joint within the range of safe zone(S) shown as FIG. 9.

In some embodiments, in order to increase the accuracy of installing position and orientation of the acetabular component, the visual marker 124 may also be attached on the impactor 140. In some embodiments, the visual marker 124 may be attached on the impactor 140 by a clamp, Velcro, taps, and the like. In some embodiments, the dimensions of the impactor 140 and a position or orientation of the visual marker 124 thereon may be unknown and need to be calibrated with the calibration procedure as described in FIG. 5.

Returning to refer FIG. 7B, after the safe zone has been determined (S301), the visual marker 124 attached on the impactor 140 may also be recognized by the one or more tracking cameras (S302). The position and orientation difference between the impactor 140, especially the acetabular component thereon, and the visual marker 121 installed on the locator may be calculated by the processing module to establish spatial conversion relationship between the impactor and the visual marker 121 installed on the locator (S303). Based on the spatial conversion relationship of the impactor 140 and the safe zone compared with the visual marker 121 installed on the locator, the relative position and orientation between the safe zone and the impactor 140 may be identified (S304). More specifically, the forward vector of the impactor 140 identified through the calibration procedure may be used to identify the relative position and orientation to the safe zone by compared with the local coordinate system of the pelvis (e.g., the abduction angle and the anteversion angle). The impactor 140 with acetabular component may be tracked and guided to align the safe zone (S305). The relative position and orientation between the safe zone and impactor 140 are used to guide surgical placement of the acetabular component trough AR or VR display into the socket at a desired position and angle per medical requirement for the patient. The relative position and orientation of inserted acetabular component and leg length may also be measured and calculated to check results are satisfying (S306).

Referring to FIG. 9, which is a schematic view of displayed images of the surgical navigation system, in accordance with an embodiment of the present disclosure. In the preferent embodiment, the landmarks of the right ASIS (A), the left ASIS (B), and the pubic symphysis (C) of the patient may be registered by the tip (P) the registration pointer 130 to determine the local coordinate system of pelvis that includes size of the pelvis size and the position and orientation of anterior pelvic plane. After the center of the hip joint (D) are determined and tracked, the safe zone(S) may be defined as the range with specific degree of abduction (inclination) angle (θ1) and specific degree of anteversion angle (θ2) with the center of the hip joint as the center point for inserting the acetabular component. In some embodiments, the center point for inserting the acetabular component may be the center of the hip joint plus the thickness of the acetabular component.

The embodiments shown and described above are only examples. Many details are often found in the art. Therefore, many such details are neither shown nor described herein. Even though numerous characteristics and advantages of the present disclosure have been set forth in the foregoing description, together with details of the structure and function of the present disclosure, the present disclosure is illustrative only, and changes may be made in the details. It will therefore be appreciated that the embodiment described above may be modified within the scope of the claims.

What is claimed is:

1. A surgical navigation system, comprising:
a head-mounted device, comprising:
a sensor module, comprising at least one tracking camera;
a processing module, connected to the sensor module; and
a display module, connected to the processing module, and comprising a display generator; and
a plurality of visual markers, recognized and tracked individually by the tracking camera;
wherein, three-dimensional position and orientation of each of the plurality of visual markers is recognized and tracked by the tracking camera, and then the processing module calculates spatial conversion relationship between each of the plurality of visual markers based on the three-dimensional position and orientation to create a local coordinate system, and then the display module generates a virtual image based on the local coordinate system through the display generator.

2. The surgical navigation system of claim 1, wherein one of the visual markers is used as a positioning reference that includes a calibration part, and the positioning reference is fixed on body of a patient or stays still when the surgical navigation system is used for a medical procedure.

3. The surgical navigation system of claim 2, further comprising a registration pointer that is attached with at least one visual marker and includes a registering part.

4. The surgical navigation system of claim 3, wherein when the registering part of the registration pointer points the calibration part of the positioning reference, the processing module calculates spatial conversion relationship between the registration pointer and the positioning reference based on the three-dimensional position and orientation of the visual markers to calibrate a distance between the registering part and the visual marker on the registration pointer.

5. The surgical navigation system of claim 4, wherein the registration pointer is used to register three-dimensional position and orientation of one or more landmarks on the patient.

6. The surgical navigation system of claim 4, wherein the registration pointer is used to calibrate a distance between a surgical instrument and a visual marker thereon through pointing boundary of the surgical instrument with the registering part.

7. The surgical navigation system of claim 6, wherein the surgical instrument is an impactor with an acetabular component for hip replacement surgery, and the registration pointer is used to calibrate a distance between the acetabular component of the impactor and the visual marker thereon through pointing two ends of the impactor with an acetabular component with the registering part.

8. The surgical navigation system of claim 4, wherein the medical procedure is selected from the group consisting of hip replacement surgery, knee replacement surgery, corrective osteotomy for malunion of an arm bone, distal femoral and proximal tibial osteotomy, peri-acetabular osteotomy, elbow ligament reconstruction, knee ligament reconstruction, ankle ligament reconstruction, shoulder acromioclavicular joint reconstruction, total shoulder replacement, reverse shoulder replacement, total ankle arthroplasty.

9. A method of using the surgical navigation system of claim 3 to assist a hip replacement surgery, comprising:
fixing a first visual marker as the positioning reference on the patient;
attaching a second visual marker on the registration pointer;
recognizing the first and the second visual markers individually and pointing the calibration part of the positioning reference by the registering part of the registration pointer points to calibrate a distance between the registering part and the visual marker on the registration pointer;
pointing one or more landmarks of pelvis of the patient by the registering part of the registration pointer to register three-dimensional position and orientation of the one or more landmarks;
defining a local coordinate system based on the three-dimensional position and orientation of the one or more landmarks;
attaching a third visual marker on femur of the patient;
recognizing the third visual markers and move the femur of the patient horizontally and vertically to determine a center of a hip joint; and
defining a safe zone for being inserted with an acetabular component based on the local coordinate system and the center of the hip joint.

10. The method of claim 9, wherein the one or more landmarks comprises a left anterior superior iliac spine (ASIS), a right ASIS, and a pubic symphysis.

11. The method of claim 10, wherein the local coordinate system is related to a pelvis size or position and orientation of an anterior pelvic plane.

12. The method of claim 9, further comprising:
attaching a fourth visual marker on an impactor with the acetabular component; and
recognizing the second and the fourth second visual markers individually and point two ends of the impactor with an acetabular component by the registering part of the registration pointer points to calibrate the distance between the acetabular component of the impactor and the fourth visual marker.

13. The method of claim 12, further comprising: tracking and guiding the impactor with the acetabular component to align the safe zone.
